(19) United States
(12) Patent Application Publication
(10) Pub. No.: US 2014/0275810 A1
(43) Pub. Date: Sep. 18, 2014

(54) SENSOR MOUNT

(71) Applicant: InnerOptic Technology, Inc., Hillsborough, NC (US)

(72) Inventors: Kurtis Keller, Hillsborough, NC (US); Caroline Green, Carrborro, NC (US); Sharif Razzaque, Chapel Hill, NC (US); Andrei State, Chapel Hill, NC (US)

(73) Assignee: InnerOptic Technology, Inc., Hillsborough, NC (US)

(21) Appl. No.: 14/212,184

(22) Filed: Mar. 14, 2014

Related U.S. Application Data
(60) Provisional application No. 61/790,765, filed on Mar. 15, 2013.
(60) Provisional application No. 61/869,313, filed on Aug. 23, 2013.

(51) Int. Cl. A61B 5/00; A61B 17/34; A61B 5/06

(52) U.S. Cl. CPC A61B 5/6848; A61B 5/065; A61B 17/3478
USPC 600/300

(57) ABSTRACT

A sensor mount that can be coupled to the shaft of a medical device can include an elongated shaft. The elongated shaft can include a lumen for coupling to the shaft of the medical device and a pose sensor for providing position and orientation information regarding the medical device. In some cases, the sensor mount can include a handle coupled to the elongated shaft and the medical device.

### PRIORITY CLAIM

The present application claim priority benefit to U.S. Prov. App. Nos. 61/790,765, filed Mar. 15, 2013 and U.S. Prov. App. No. 61/869,313, filed Aug. 23, 2013, each of which is hereby incorporated by reference in its entirety.

### BACKGROUND

Many medical procedures require a needle, needle-like device, or medical device, to be inserted into the patient through a trocar. Some of these devices benefit from the addition of pose (position and/or orientation) tracking, directing, and/or predicting where the device can, or should, go inside a body. Typically, when a pose sensor is used with a medical device, it is embedded into the medical device. However, medical devices often do not have sufficient space for a pose sensor and/or need to be modified in order to accommodate the pose sensor. Furthermore, regulatory approval is often required to modify the medical device to accommodate the sensor, which can be both time consuming and expensive. In addition, placing a pose sensor near the needle handle often results in inaccurate pose information due to the natural flexibility of the medical device. For example, some medical devices can deflect 20-30 degrees once inserted, which can result in accuracy variations from 50-100 mm or more, depending on the distance between the pose sensor and the distal end of the medical device. In many medical procedures this variation is not acceptable.

### SUMMARY

One or more embodiments of a sensor mount are described that allow a user to couple one or more pose (position and/or orientation) sensors to an existing medical device. The medical device and the sensor mount can then be inserted into the patient as an assembly. In some embodiments, the sensor is not designed into the medical device by the manufacturer, but is coupled to the medical device prior to use by a medical practitioner (e.g., doctor, nurse, technician, assistant, caretaker, etc.).

### BRIEF DESCRIPTION OF THE DRAWINGS

FIG. 1A is a rendering of an embodiment of a sensor mount.

FIG. 1B is a rendering of a distal end of a sensor mount.

FIG. 1C is a rendering of a portion of the shaft of a medical device and a sensor mount including a sleeve.

FIG. 1D is a rendering of a handle of a sensor mount.

FIG. 1E is a rendering of a handle of a sensor mount coupled with a medical device.

FIG. 2 is a diagram of an embodiment of a sensor mount.

FIG. 3A is a rendering of an embodiment of a sensor mount.

FIG. 3B is a diagram of an embodiment of a sensor mount.

FIG. 4A is a rendering of an embodiment of a junction block coupled with a medical device.

FIG. 4B is a diagram of a sensor mount coupled to a medical device and a junction block including a spring.

FIG. 5 is a diagram of embodiments of a cross section of a wire.

### DETAILED DESCRIPTION

As described in greater detail below, a sensor mount can be used to couple one or more pose sensors to an existing medical device. The sensor mount can be implemented as a sheath or sleeve, clip-on, or other configuration. The pose sensor, described in greater detail in U.S. application Ser. No. 13/753,274, filed Jan. 29, 2013, now issued as U.S. Pat. No. 8,670,816, (the '274 Application) and U.S. application Ser. No. 13/014587, filed Jan. 26, 2011, now issued as U.S. Pat. No. 8,641,621 (the '587 Application), each of which is incorporated by reference herein in its entirety, can report pose with 3, 5 or 6 degrees of freedom, etc., and can be magnetic, electrostatic, proximity, optical, inertial, sonic, etc. The pose sensor can be located within or on the outside of the sensor mount and can therefore provide position and orientation information regarding the sensor mount (and the medical device when the medical device is coupled to the sensor mount). In some embodiments, the sensor can be wrapped around the elongated shaft.

The medical device(s) can include, but is not limited to, ablation needles (e.g., radiofrequency ablation needles, microwave ablation needles), biopsy needles, brachytherapy needles, electroporation electrodes, solid needles, hollow needles, aspiration needles, injection needles, flexible needles, ultrasound needles, etc., other needle-like devices, and/or laparoscopic instruments such as graspers, cameras, ultrasound transducers, staplers, electro cautery instruments, harmonic shears, etc.

Reference throughout the specification is made to distal and proximal portions of the sensor mount. The terms proximal and distal are used herein from the perspective of a handle of a medical device when used with the sensor mount. For example, the tip of the sensor mount can be referred to as the distal end of the sensor mount and the handle of the sensor mount can be referred to as the proximal end of the sensor mount.

FIG. 1A is a rendering of an embodiment of a sensor mount 100. In some embodiments, the sensor mount 100 can include an elongated shaft 102 and a handle 104. The elongated shaft 102 and/or handle 104 can be made as an integrated sensor mount 100 and/or can be coupled together (e.g., by using glue, epoxy, tape, or other adhesive, compression nut, screws, nails, clamp, welding, soldering, compression, etc.). Furthermore, the elongated shaft 102 and the handle 104 can be molded and/or manufactured from the same or different material. For example, the elongated shaft 102 and/or the handle 104 can be made of nylon, stainless steel, titanium, aluminum, polyimide, Kevlar, plastic, polymer, composite material, or other rigid or semi-rigid material, as desired. In some embodiments, the elongated shaft 102 can be flexible to allow the medical device to flex or bend. In certain embodiments, the material used is a medical-grade, non-magnetic material. Any combination of the above-described embodiments can be used as desired.

In some embodiments, a distal portion of the handle 104 can interface with a proximal portion of the elongated shaft 102. For example, an indentation in the handle 104 can interface with a proximal end of the elongated shaft 102. In some embodiments, the proximal end of the elongated shaft 102 can be adhered to the handle (e.g., by using glue, epoxy, tape, or other adhesive, compression nut, screws, nails, clamp, welding, soldering, compression, etc.).

In some cases, the length and dimensions of the elongated shaft 102 can be based at least in part on the dimensions of the medical device used with the sensor mount 100, other medical devices used during the medical procedure, and/or physiological characteristics of the patient. For example, the width and/or or cross-sectional dimensions of the elongated shaft 102 can be based at least in part on the cross-sectional dimension of the medical device that is fitted with the sensor mount 100, an introducer, and/or a trocar (or other medical device) that is used during the medical procedure. For example, in some embodiments, the cross-sectional area of the elongated shaft 102 is larger than the cross-sectional area of the medical device, but smaller than the cross-sectional area of the trocar, the introducer, and/or an area threshold (e.g., the area corresponding to an incision or entry point in the patient). In this manner, the elongated shaft 102 can fit around the medical device, while remaining small enough to be inserted into the patient (e.g. via the trocar, the introducer, by itself, etc.). In certain embodiments, the cross-sectional area of the elongated shaft 102 is determined based at least in part on a size of anatomical features of the patient. In this way, the sensor mount 100 can be used without causing damage to the patient.

In some embodiments, the elongated shaft 102 fits all the way around at least a portion of the medical device. For example, the elongated shaft can enclose a cross-section of the medical device shaft like a sleeve or sheath. In such embodiments, the medical device can be threaded through the elongated shaft 102. In some embodiments, the elongated shaft 102 can include a seam that allows the elongated shaft to be snapped onto the medical device shaft. In certain embodiments, the elongated shaft 102 can fit around a portion of the medical device. For example, the cross-section of the elongated shaft 102 can be ‘C’ shaped and fit around half, three-quarters, or more (or less), of the cross-section of the shaft of the medical device (other dimensions can be used as desired). In some embodiments, the elongated shaft 102 can be configured to snap onto the medical device or otherwise be coupled and/or adhered to the medical device. In certain embodiments, the medical device can be threaded through the elongated shaft 102.

In some embodiments, the elongated shaft can include a sheath portion and a hinged portion. The sheath portion can include half or more of the cross-section of the elongated shaft and can be snapped onto the medical device and/or the medical device can be threaded through the sheath portion. The hinged portion can include a pivot, hinge-pin, or living hinge. Once the medical device is coupled with the sheath portion, the hinged portion can be closed so that the elongated shaft 102 fits around the medical device shaft. In some embodiments, the hinged portion can provide space for the pose sensor and sensor wires.

In some embodiments, the location of the elongated shaft 102 and/or the location to which the elongated shaft 102 extends can be determined based at least in part on the medical device used with the sensor mount 100. For example, the medical device can have an active portion (e.g., heating portion, heating zone, ablation zone, electrode, cryo zone, seed placement (bracheotherapy), biopsy, injection, marker placement, electroporation, carterization, morselation, etc.) and a passive portion. In certain embodiments, the elongated shaft 102 can extend along the medical device shaft in the passive portion and can end at, or prior to, the active portion. In some embodiments, the elongated shaft 102 does not extend along the active portion of the medical device. In this way, the elongated shaft 102 can avoid interfering with the active portion of the medical device, while providing a location to place the pose sensor closer to the distal end of the medical device than is otherwise possible. For example, in some embodiments, the elongated shaft 102 can extend along the medical device shaft to an electrode of the medical device.

In certain embodiments, the distal end of the elongated shaft 102 ends at or prior to the electrode. In some embodiments, the distal end of the elongated shaft 102 extends to a heat or ablation zone of the medical device. In certain embodiments, the distal end of the elongated shaft 102 ends at, or prior to, the heat or ablation zone. In some cases, the elongated shaft 102 can extend along the entire length of the medical device and/or extend beyond the length of the medical device. In certain embodiments, the elongated shaft 102 does not extend along the entire length of the shaft of the medical device. In some embodiments, the elongated shaft 102 is located at or near a distal end of the medical device, such as near a transducer of a laparoscopic ultrasound, as illustrated in FIG. 4B.

In some embodiments, as illustrated in FIGS. 1B and 1C, a distal end of the elongated shaft 102 can be sharpened, beveled, and/or angled in order to increase the ease at which the sensor mount 100 can be inserted into a patient. The end can be angled on one side, as illustrated in FIG. 1C, or on multiple sides (e.g., to create a point), an embodiment of which is illustrated in FIG. 1B, as desired. In certain embodiments, a proximal end of the elongated shaft 102 can be sharpened, beveled, and/or angled as desired.

In the illustrated embodiment of FIGS. 1A-1C, the elongated shaft 102 can include two lumens: a medical device lumen 106 and a sensor lumen 108. However, it will be understood that the elongated shaft 102 can include fewer or more lumens as desired. For example, in some embodiments, the elongated shaft 102 can include three or more lumens. The additional lumens can be medical device lumens and/or sensor lumens, as desired. For example, in some instances, the medical device lumen 106 can be in the center, or approximately in the center of the elongated shaft 102 (e.g. center of the cross-section), and two sensor lumens can be equidistant from the medical device lumen 106 and on opposite sides of the medical device lumen 106. Additional sensor and/or medical device lumens can be used as desired. In certain embodiments, the elongated shaft 102 can include one lumen, such as the medical device lumen 106. In such embodiments, one or more sensors and/or corresponding wires can be embedded in, wrapped around, and/or adhered to the elongated shaft 102, as desired. Furthermore, in some embodiments, the medical device lumen 106 is larger or smaller than the sensor lumen 108. In certain embodiments, the medical device lumen 106 and the sensor lumen 108 are the same size, or approximately the same size.

In some embodiments, the medical device lumen 106 can extend along the entire length of the elongated shaft 102. The cross-sectional dimensions (e.g., circumference, internal cross-sectional perimeter) of the medical device lumen 106 can be manufactured to correspond to the cross-sectional dimensions of the medical device. For example, if the cross-section of the medical device is circular, the cross-section of the medical device lumen 106 can be circular and/or semi-circular, etc. Similarly, the medical device lumen 106 can be manufactured to have a cross-section that is square, rectangle, oval, obround, triangle, quadrilateral, hexagonal, etc. and/or complements a medical device that is square, rectangle, oval, obround, triangle, quadrilateral, hexagonal, etc., as desired.

In addition, in some embodiments, the medical device lumen 106 can be made to be equal, or approximately equal, to the cross-sectional dimensions of the medical device. In certain embodiments, the medical device lumen can be slightly smaller, and/or slightly larger to the cross-sectional dimensions of the medical device. If made approximately equal or slightly smaller, the elongated shaft 102 can have some flexibility that enables the medical device lumen 106 to adjust to the size of the medical device. In certain embodiments, for example, such as when the elongated shaft 102 does not fit all the way around the medical device, the cross-sectional dimensions of the medical device lumen 106 can be configured to correspond at least a portion of the cross-sectional dimensions of the medical device (e.g., half, three-quarters, etc.). In this way the elongated shaft 102 can be clipped and/or snapped onto the medical device.

In some embodiments, the dimensions of the medical device lumen 106 can vary along the length of the elongated shaft 102. In some cases, the variation can be due to a medical device that has varying dimensions (e.g., wider and/or narrower at different portions), and in certain instances the variation can be with respect to the dimensions of the medical device. For example, if the dimensions of the medical device are relatively constant across its shaft, or if the dimensions of the medical device vary across its shaft, the dimensions of the medical device lumen 106 can vary with respect to the dimensions of the medical device shaft. In this way, the elongated shaft 102 can be configured to fit more snugly, or tightly, with some parts of the medical device than others. The portions that fit more tightly can be used to maintain the position and orientation of the sensor mount with respect to the medical device.

Furthermore, as shown in FIG. 1C, in some embodiments, an adapter sleeve 107 can be used to adapt the size of the medical device lumen 106 to the size of the medical device shaft 109. For example, if the diameter of the medical device lumen 106 is 10 mm, and the diameter of the medical device shaft 109 is 6 mm, the adapter sleeve 107 can be used to adapt the medical device lumen 106 to fit with the smaller medical device shaft 109. In some embodiments, the adapter sleeve 107 is made from the same or different material as the elongated shaft 102. For example, in some embodiments, the elongated shaft 102 can be made of nylon and the adapter sleeve 107 can be made of Teflon, etc.

The sensor lumen 108 can, in some embodiments, extend along the length of the elongated shaft 102. In certain embodiments, the sensor lumen 108 can extend along a portion of the length of the elongated shaft 102. For example, in some embodiments, the sensor lumen 108 can be implemented as a cavity in a distal, medial, and/or proximal portion of the elongated shaft. In certain embodiments, the sensor lumen 108 can be implemented as a protrusion from a portion of the elongated shaft 102. In some embodiments, the sensor lumen 108 extends along a portion of the elongated shaft 102 that is distal to the handle and does not extend along the entire length of the elongated shaft 102.

The cross sectional dimensions of the sensor lumen 108 can, in some embodiments, be manufactured to correspond to the dimensions of the sensor used with the sensor mount 100. For example, the cross sectional dimensions of the sensor lumen 108 can be configured to correspond to a pose sensor that is used with the sensor mount 100. In some embodiments, the cross-sectional dimensions of the sensor lumen 108 can be larger than the pose sensor so that the sensor can be coupled to the sensor lumen 108. The sensor can be coupled to the lumen in a variety of ways, such as, but not limited to, by using glue, epoxy, tape, or other adhesive, compression nut, screws, nails, clamp, welding, soldering, compression, etc., as desired. In some embodiments, one or more portions of the sensor lumen 108 can be indented (e.g., such as by crimping at least a portion of the elongated shaft 102) to maintain the sensor in a desired location. Similar to the adapter sleeve 107, a sensor lumen adapter sleeve can be used to adapt the size of the sensor lumen 108 with the sensor.

When the sensor includes wires, the wires can be located within the sensor lumen 108, embedded within the elongated shaft 102 and/or located within the medical device lumen 106. In some embodiments, the wires can be located within grooves in the medical device lumen 106 and/or the sensor lumen 108. In certain embodiments, the sensor, or portions thereof, can be wrapped around the elongated shaft 102, the medical device lumen 106 and/or the sensor lumen 108.

In some embodiments, openings on one or both ends of the sensor lumen 108, as well as the sensor, can be filled and/or covered. In some embodiments, the entire sensor lumen 108 can be filled. For example, after the sensor is located within the sensor lumen 108, one or both openings at the end of the lumen can be filled with epoxy, covered with a cap, or otherwise covered and/or filled. Covering the distal end of the sensor lumen can prevent tissue from the patient from interacting with the sensor. Covering the proximal end of the sensor lumen 108 can make it easier to sanitize the sensor mount 100.

With reference to FIG. 1D, the handle 104 can include a medical device lumen 110, grooves 112, 114, a cavity 116, and complementing medical device contours 118. In some cases, the handle can also include one or more protrusions, recesses, or grips for user comfort, such as the trigger-like protrusion 126, illustrated in FIG. 1E. Furthermore, in some embodiments, the handle 104 can include one or more pose sensors to provide position and orientation data of the handle 104.

In some embodiments, the handle 104 can be coupled and/or adhered to the medical device (e.g., by using glue, epoxy, tape, or other adhesive, compression nut, screws, nails, clamp, welding, soldering, compression, etc.). In addition, as described above, with reference to the elongated shaft 102, the handle 104 can, in some embodiments, enclose at least a portion of the medical device (e.g., fit around a cross-section of the medical device shaft like a sheath), and in certain embodiments, the handle 104 may not enclose the medical device (e.g., fit around less than 100% of the cross-section of the medical device shaft (as illustrated by the shade 120 of the handle 104 in FIG. 1E), such as in a ‘C’ shape, described previously. In such embodiments, the handle 104 can snap onto the medical device shaft and/or the medical device can be threaded through the handle 104, as desired.

The medical device lumen 110 can correspond to the medical device lumen 106 of the elongated shaft 102, such that the medical device shaft can pass through the medical device lumen 110 of the handle 104 and into the medical device lumen 106 of the elongated shaft 102. Although not visible in FIG. 1C, it will be understood that the handle can include a sensor lumen corresponding to the sensor lumen 108 of the elongated shaft 102. In this way, wires from the sensor in the sensor lumen 108 can pass through the handle and into the cavity 116, as described below.

The sensor wires from the sensor in the sensor lumen 108 can be located within the groove 112, thereby enabling the sensor wires to reach the cavity 116. Cables for the medical device system, such as the navigation systems described in greater detail in the '274 and '587 Applications, previously incorporated herein by reference, can be located within the groove 114. Although illustrated on a proximal portion of the handle 104, it will be understood that the grooves 112 can be located on a distal and/or proximal portion of the handle 104, as desired.

In some embodiments, the sensor wires and the medical device system cables can be different. For example, the medical device system cables can be larger, more robust, sheathed, and/or more protected. In certain embodiments, the sensor wires and the medical device system cables can be the same. For example, in such embodiments, the wires from the sensor can be long enough and robust enough to extend from the sensor to the medical device system.

The cavity 116 can be used to interface the wires from the sensor with the medical device system cables, and although not illustrated in FIG. 1C, can include a cover to cover the sensor wires and the medical device system cables. For example, the sensor wires and the medical device system cables can be coupled together within the cavity 116 (e.g., crimped, soldered, etc.) and covered by the cover. In some embodiments, the cavity 116 can be filled with epoxy to decrease the strain on the sensor wires and the medical device system cables. It will be understood that a variety of techniques can be used to decrease the strain on the sensor wires and/or the medical device system cables. For example, pegs or hooks can be used. Furthermore, although the cavity 116 is located on a proximal portion of the handle 104 in the illustrated embodiment, it will be understood that the cavity 116 can be located on a distal portion and/or a proximal portion, as desired. In addition, in some embodiments, the handle can omit the cavity 116. For example, in some embodiments, the coupling between the sensor wires and the medical device system cables can be embedded within the handle 104, etc. Any combination of the above-described embodiments can be used as desired.

The complementing medical device contours 118 can be used to interface the handle 104 with the medical device 103. In some embodiments, the contours 118 can correspond to contours of the medical device 103. For example, if the medical device includes a protrusion at a particular location, the contours 118 can include an indentation at the particular location, and vice versa. In certain embodiments, the contours 118 can be used to couple the handle 104 to the medical device. For example, the contours 118 can be used to snap, clamp, clip, compress, etc. the handle 104 onto a portion of the medical device 103. In some embodiments, at least a portion of the contour 118 can include material to better adhere to the medical device (e.g., rubber or other flexible material that can have a high coefficient of friction when interfacing with the medical device, etc.). Although, illustrated in FIG. 1D as having one interfacing contour 118, it will be understood that that handle 104 can include multiple interfacing contours to interface the handle 104 with the medical device 103, as desired. For example, as illustrated in FIG. 1E, the handle 104 can include multiple contours 118, etc.

FIG. 2 is a diagram of an embodiment of the sensor mount 100. In the illustrated embodiment of FIG. 2, the elongated shaft 102 of the sensor mount 100 includes a medical device shaft 102A and a sensor shaft 102B. The medical device shaft 102A and the sensor shaft 102B can be formed as an integrated shaft 102 and/or can be coupled and/or adhered together, as desired. In some embodiments, the medical device lumen 106 can be included in the medical device shaft 102A, and the sensor lumen 108 can be included in the sensor shaft 102B. In certain embodiments, the sensor, or at least a portion thereof, can be wrapped around the medical device shaft 102A and/or the sensor shaft 102B. In addition, the illustrated embodiment of FIG. 2 shows the cover 122 to the cavity 116 and the cavity 116 located on a distal portion of the handle 104.

In some embodiments, the medical device shaft 102A or the sensor shaft 102B can be sharpened, or angled as desired. For example, in some cases the sensor shaft 102B can be sharpened, and the medical device shaft 102A is not, or vice versa. In certain embodiments both the medical device shaft 102A and the sensor shaft 102B are sharpened or are not sharpened, as desired.

Furthermore, in the illustrated embodiment of FIG. 2, the handle 104 includes various contours 118 to interface with the medical device. For example, the protrusion 124 of the handle 104 can interface with an indentation of the medical device. As such, the handle 104 can be coupled with the medical device (e.g., snapped onto, clamped, compressed, etc.). It will be understood that additional and/or different protrusions/indentations can be included as desired.

FIG. 3A is a diagram of an embodiment of a sensor mount 300. In the illustrated embodiment of FIG. 3A, the sensor mount 300 is implemented as a band-like clip. The sensor mount 300 can include an elongated shaft 302 for clamping and/or securing the sensor mount 300 to a medical device and a cover 314. In some embodiments, the elongated shaft 302 can include a clamping portion 303, a medical device lumen 304, a sensor cavity 306, an interface cavity 308, groove 310, and/or calibration indicators 312. However, it will be understood that some embodiments can include fewer or more portions as desired. For example, in some embodiments, the elongated shaft 302 does not include a clamping portion 303, interface cavity 308, groove 310, and/or calibration indicators 312, etc. Furthermore, as discussed previously, a distal and/or proximal end of the sensor mount 300 (e.g., the elongated shaft 302, can be sharpened, angled, and/or beveled, as desired.

The length and dimensions of the elongated shaft 302, as well as the materials used to make the elongated shaft 302 can be similar to those described previously with reference to the elongated shaft 102. In some embodiments, the elongated shaft 302 is smaller (e.g., shorter) than the elongated shaft 102 and extends across only a portion of the length of the medical device shaft. For example, the elongated shaft 302 can be located near (or proximal to) a distal portion of the medical device, such as, near (or proximal to) a transducer of a laparoscopic ultrasound device (illustrated in FIG. 4B as transducer 454). In certain embodiments, the elongated shaft 302 does not extend across the entire length of the medical device shaft.

In some embodiments, the location of the sensor mount 300 is based at least in part on one or more features of the medical device, such as, but not limited to, a needle guide, offset, indentation, ultrasound pad, etc. In certain embodiments, the sensor mount 300 can be located on the medical device such that it aligns with one or more features of the medical device. For example, the sensor mount 300 can be aligned such that the elongated shaft 302 is coupled between two features of the medical device shaft, such as an indentation and an ultrasound pad. In this way, the location of the sensor mount can be the same or approximately the same each time it is placed on the medical device.

In some embodiments, the elongated shaft 302 can include a clamping portion 303. The clamping portion can be a portion of the elongated shaft 302 that has a smaller cross-sectional area than other portions of the elongated shaft 302, and can be used to more firmly fit the sensor mount 300 to the medical device. In some embodiments, the clamping portion 303 is configured so that it corresponds to a feature of the medical device.

The medical device lumen 304 can be implemented similar to the medical device lumen 106, described previously. As described previously, the medical device lumen 304 can be shaped to complement the shape of the medical device. In some embodiments, the medical device lumen 304 can be circular, semi-circular, oval, square-like, rectangular, etc. It will be understood that in embodiments where the elongated shaft 302 does not fit all the way around the medical device, the medical device lumen 304 can include the corresponding shape (or portions thereof) in order to be coupled with the medical device.

The sensor cavity 306 can be used to place the pose sensor, similar to the placement of the pose sensor in the sensor lumen 108, described previously. In some embodiments, once the pose sensor is located in the sensor cavity 306, an epoxy or other material is placed in the sensor cavity 306 to protect the sensor from exposure. In certain embodiments, the sensor cavity 306 can be implemented similar to the sensor lumen 108, described previously with reference to the sensor mount 100. Furthermore, in some embodiments, the sensor mount 300 can omit the sensor cavity 306, and the sensor can be embedded into the elongated shaft 302.

The interface cavity 308 can be used to interface the wires from the sensor with wires from a cable. Once the wires are electrically coupled (e.g., soldered, crimped, twisted, tied etc.), epoxy, or other material, can be applied to protect the wires from exposure, as well as to relieve strain. The cover 314 can be used to protect the interface cavity 308 and its contents from exposure. In some embodiments, the sensor mount 300 can omit the interface cavity 308 and/or cover 314, and the sensor wires can extend from the sensor to the navigation system and/or can be embedded into the elongated shaft 302.

The groove 310 can be used to position the wires from the sensor. In addition, epoxy can be applied to relieve strain on the wires in the groove 310. However, as described previously, strain can be relieved in a variety of ways. The calibration indicators 312 can be used to calibrate the location and orientation of the sensor within the sensor mount 300 and/or calibrate the location of the sensor mount 300 on the medical device. In certain embodiments, the calibration indicators 312 can be used at manufacturing. Furthermore, in some embodiments, the calibration indicators 312 can be indicated on a display by the medical device system and used by the user to identify the location of the sensor mount 300. In some embodiments, the groove 310 can be omitted. For example, in some instances, the sensor wires can be embedded in the elongated shaft 302 and/or the sensor wires can extend along the side of the elongated shaft 302. Any combination of the aforementioned embodiments can be used as desired.

As described previously with reference to the elongated shaft 102, the elongated shaft 302 can include a sheath portion and a hinged portion. The sheath portion can include half or more of the cross-section of the elongated shaft and can be snapped onto the medical device and/or the medical device can be threaded through the sheath portion. The hinged portion can include a pivot, hinge-pin, or living hinge. Once the medical device is coupled with the sheath portion, the hinged portion can be closed so that the elongated shaft 302 fits around the medical device shaft. In some embodiments, the hinged portion can provide space for the pose sensor and/or sensor wires. An illustration of an embodiment of the elongated shaft 302 with a sheath portion 352 and a hinged portion 354 is provided in FIG. 3B. Any combination of the above reference embodiments can be used as desired. Furthermore, any combination of the components described above with reference to FIG. 3A can be used with the illustrated embodiment of FIG. 3B as desired.

FIG. 4A is a rendering of an embodiment of a junction block 402 coupled with a medical device 452. In some embodiments, the sensor mount 300 can be used in conjunction with a junction block 402 located on or near the medical device handle 450. In certain embodiments, the junction block 402 can include a medical device lumen similar to the medical device lumen 304, described previously. In some embodiments, the junction block 402 can be coupled with and/or adhered to the medical device shaft 452 and/or the medical device handle 450. In some embodiments, the junction block 402 fits around the cross-section of the medical device shaft 452. In certain embodiments, the junction block 402 fits around a portion of the cross-section of the medical device shaft 452. In some embodiments, the junction block 402 can include a sheath portion and a hinge portion as described previously with reference to the elongated shaft 302. In certain embodiments, the junction block 402 can include a pose sensor.

The junction block 402 can receive the sensor wires from the sensor coupled to the sensor mount 300 and can further include a cavity similar to the cavity 116, described previously with reference to the sensor mount 100. For example, the junction block cavity can be used to interface the wires from the sensor with medical device system cables 404 and relieve strain on the wires. In addition, the junction block 402 can include a cover to cover the sensor wires and the medical device system cables 404 residing in the junction block cavity.

FIG. 4B is a diagram of a sensor mount 300 coupled to the shaft 452 of a medical device, and a junction block 402 including a spring 420. As illustrated in FIG. 4B, the sensor mount 300 can be coupled to the shaft 452 of the medical device, and wires 422 from the sensor coupled to the sensor mount 300 can extend to the junction block 402. In certain embodiments, the junction block 402 can include a spring 420 that is coupled with the sensor wires 422. In some embodiments, such as when the sensor mount 300 is used with a medical device with an articulated head, the spring 420 can be used to recoil the sensor wires 422 when there is slack. For example, when the head 456 of the medical device is articulated, the spring 420 can be stretched. After the head 456 is moved back, the spring 420 can be used to recoil the sensor wires 422. In some embodiments, the sensor mount 300 and/or the junction block 402 can be used with a sterile drape. In certain embodiments, a sleeve is used to keep the sensor wires close to the medical device.

In certain embodiments, one or more indicators can be included on a portion of the elongated shaft of the sensor mount 300, the shaft 452 of the medical device that is proximal to the handle 450 of the medical device, and/or the handle 450 of the medical device. The indicators can be used to help the user orient the medical device and can include, but are not limited to a fin, arrow, divot, label, detent, and/or a button, etc.

FIG. 5 is a diagram of embodiments of a cross section of sensor wires. In some embodiments, the wires from the sensor mount 300 that extend down the shaft of the medical device can be configured in order to reduce the amount of insufflator gasses and liquids that exit the patient. For example, in some embodiments, the wires from the sensor mount 300 can be adhered to the medical device (e.g., by using glue, epoxy, tape, or other adhesive, etc.). In certain embodiments, the cross section of the wires and/or wire cover or casing can be altered.

In some embodiments, by altering the cross-sectional shape of the wire and/or wire cover or casing, the wire can more easily be fitted and/or adhered to the medical device and/or a cable. For example, Wire 502 illustrates an embodiment of the cross-section of a typical wire. Wire 504 illustrates an embodiment of the cross-section of a wire with a curved bottom 510 to follow the contours of a medical device. Wire 506 illustrates an embodiment of the cross-section of a wire with a cut-out 510 so it can be pressed over an existing wire or cable. Wire 508 illustrates an embodiment of the cross-section of a wire with a flat bottom 516 to lie against the medical device and to make a light adhesive or sticker easier to apply. As can be seen, the wires 504-508 can provide a larger surface area to adhere to the medical device and/or a cable.

Reference throughout this specification to “some embodiments,” “certain embodiments,” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment and may refer to one or more of the same or different embodiments. Furthermore, the particular features, structures or characteristics may be combined in any suitable manner, as would be apparent to one of ordinary skill in the art from this disclosure, in one or more embodiments.

As used in this application, the terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Similarly, it should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single foregoing disclosed embodiment.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the features illustrated may be made without departing from the spirit of the disclosure. As will be recognized, certain embodiments of the inventions described herein may be embodied within a form that does not provide all of the features and benefits set forth herein, as some features may be used or practiced separately from others.

1. A sensor mount comprising:
a handle configured to couple to a medical device; and
an elongated shaft configured to couple to at least a portion of a shaft of the medical device, the elongated shaft comprising:
a medical device lumen for coupling to the shaft of the medical device, and
a pose sensor lumen including a pose sensor located at a portion of the elongated shaft that is distal to the handle.

2. The sensor mount of claim 1, wherein the handle comprises a cavity for electrically coupling wires from the pose sensor to a medical device cable.

3. The sensor mount of claim 1, wherein the medical device lumen is a first medical device lumen and the handle comprises a second medical device lumen for coupling to the shaft of the medical device.

4. The sensor mount of claim 1, wherein the handle comprises one or more contours for coupling to a handle of the medical device.

5. The sensor mount of claim 4, wherein the one or more contours comprise at least one of a protrusion and indentation corresponding to a complementary at least one of an indentation and protrusion of the handle of the medical device.

6. The sensor mount of claim 1, wherein a distal end of the elongated shaft comprises a sharpened tip.

7. The sensor mount of claim 1, wherein the elongated shaft extends along the length of a passive portion of the shaft of the medical device.

8. The sensor mount of claim 1, wherein the pose sensor is a first pose sensor and the first pose sensor lumen is a first pose sensor lumen, and the elongated shaft further comprises a second pose sensor lumen for enclosing a second pose sensor located at the portion of the elongated shaft that is distal to the handle.

9. The sensor mount of claim 8, wherein the first pose sensor lumen and the second pose sensor lumen are equidistant from the center of the medical device lumen.

10. The sensor mount of claim 1, wherein the elongated shaft encloses at least a portion of a cross-section of the at least a portion of the medical device.

11. The sensor mount of claim 10, wherein the elongated shaft encloses the cross-section of the at least a portion of the medical device.

12. The sensor mount of claim 1, wherein the elongated shaft comprises a sheath portion and a hinged portion.

13. The sensor mount of claim 1, wherein the elongated shaft further comprises:
a medical device shaft comprising the medical device lumen; and
a sensor shaft coupled to the medical device shaft, the sensor shaft comprising the sensor lumen.

14. The sensor mount of claim 1, wherein the elongated shaft further comprises a sleeve located within the medical device lumen.

15. The sensor mount of claim 1, wherein the elongated shaft further comprises a sleeve located within the sensor lumen.

16. The sensor mount of claim 1, wherein at least one of the medical device shaft and the sensor shaft comprise a sharpened tip.

17. The sensor mount of claim 1, wherein a distal end of the pose sensor lumen and a proximal end of the pose sensor lumen are covered.

18. The sensor mount of claim 1, wherein the pose sensor lumen extends along the length of the elongated shaft.

19. The sensor mount of claim 1, wherein the pose sensor lumen extends along a portion of the length of the elongated shaft.

20. The sensor mount of claim 1, wherein the pose sensor lumen extends along a portion of the elongated shaft that is distal to the handle and does not extend along the entire length of the elongated shaft.

21. A sensor mount comprising:
an elongated shaft configured to couple to at least a portion of a shaft of a medical device, the elongated shaft comprising:
a lumen for coupling to the shaft of the medical device, and
a pose sensor enclosed within a sensor cavity and located at a portion of the elongated shaft that is distal to a handle of the medical device.

22. The sensor mount of claim 21, wherein the elongated shaft extends across only a portion of the length of the shaft of the medical device

23. The sensor mount of claim 22, wherein the elongated shaft is proximal to a distal end of the medical device.

24. The sensor mount of claim 21, wherein the distal end of the medical device comprises an ultrasound transducer.

25. The sensor mount of claim 21, wherein the elongated shaft further comprises a cavity for electrically coupling wires from the pose sensor to medical device system cables.

26. The sensor mount of claim 21, wherein the elongated shaft encloses the cross-section of the at least a portion of the medical device.

27. The sensor mount of claim 21, wherein the elongated shaft comprises a sheath portion and a hinged portion.
